(19) United States
(12) Patent Application Publication
(10) Pub. No.: US 2007/0027171 A1
(43) Pub. Date: Feb. 1, 2007

(54) Cycloalkanepyrrolopyridines as dp receptor antagonists

(76) Inventors: Nicolas Lachance, Pierrefonds (CA); Claudio Sturino, L'ile Bizard (CA)

Correspondence Address: MERCK AND CO., INC, P O BOX 2000, RAHWAY, NJ, 07065-0907, US

(21) Appl. No.: 10/558,909

(22) PCT Filed: Jun. 9, 2004
(86) PCT No.: PCT/CA04/00833

§ 371 Date: Dec. 1, 2005

Related U.S. Application Data
(60) Provisional application No. 60/479,192, filed on Jun. 12, 2003.

(51) Int. Cl. A61K 31/519; A61K 31/4745; C07D 487/02

(52) U.S. Cl. 514/267; 514/291; 544/250; 546/85

(57) ABSTRACT

Novel cycloalkanepyrrolopyridine derivatives are antagonists of prostaglandins, and as such are useful for the treatment of prostaglandin mediated diseases.

### BACKGROUND OF THE INVENTION

The present invention relates to compounds and methods for treating prostaglandin mediated diseases, and certain pharmaceutical compositions thereof. More particularly, the compounds of the invention are structurally different from steroids, antihistamines or adrenergic agonists, and are antagonists of the nasal and pulmonary congestion effects of D-type prostaglandins.

Two review articles describe the characterization and therapeutic relevance of the prostanoid receptors as well as the most commonly used selective agonists and antagonists: Eicosanioids: From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87. An article from T. Tsuri et al. published in 1997 in Journal of Medicinal Chemistry, vol 40, pp. 3504-3507 states that “PGD2 is considered to be an important mediator in various allergic diseases such allergic rhinitis, atopic asthma, allergic conjunctivitis and atopic dermatitis.” More recently, an article by Matsuoka et al. in Science (2000), 287:2013-7, describes PGD2 as being a key mediator in allergic asthma. In addition, patents such as U.S. Pat. No. 4,808,608 refer to prostaglandin antagonists as useful in the treatment of allergic diseases, and explicitly allergic asthma. PGD2 antagonists are described in, for example, European Patent Application 837,052 and PCT Application WO98/25919, as well as WO99/62555.

### SUMMARY OF THE INVENTION

The present invention provides novel compounds which are prostaglandin receptor antagonists; more particularly, they are prostaglandin D2 receptor (DP receptor) antagonists. Compounds of the present invention are useful for the treatment of various prostaglandin-mediated diseases and disorders; accordingly the present invention provides a method for the treatment of prostaglandin-mediated diseases using the novel compounds described herein, as well as pharmaceutical compositions containing them.

### DETAILED DESCRIPTION OF THE INVENTION

The present invention relates to compounds of formula I: 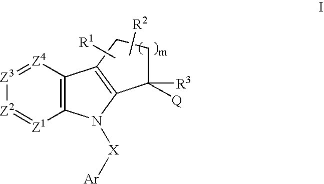
and pharmaceutically acceptable salts and hydrates thereof, wherein:
Ar is aryl or heteroaryl each optionally substituted with one to four groups independently selected from Rg;
Q is —A—Q′;
A is selected from (1) C₁₋₃alkyl optionally substituted with one to four halogen atoms or with one to two CF₃ groups, (2) O(CH₂)₁₋₂, and (3) S(O)n(CH₂)₁₋₂;
Q′ is selected from COOH, CONR^{a}R^{b}, C(O)NHSO₂R^{c}, SO₂NHR^{a}, SO₃H, PO₃H₂, and tetrazolyl;
one of Z¹, Z², Z³ or Z⁴ is N or N→O, and the others are independently selected from CH and C—Rg;
X is selected from —(CR^{d}R^{e})ₐ—W—(CR^{d}R^{e})^{b}—, phenylene, C₃₋₆ cycloalkylidene and C₃₋₆cycloalkylene, wherein a and b are integers 0-1 such that the sum of a and b equals 0, 1 or 2, and W is a bond, —SO₂—, —C(O)—, —CH(OR^{a})—, —C(O)O—, —C(O)NR^{a}—, —CR^{d}═CR^{e}— or —C═C—;
R¹ is selected from H, CN, OR^{a}, —S(O)ₙC₁₋₆alkyl and C₁₋₆alkyl optionally substituted with one to six groups independently selected from halogen, OR^{a} and —S(O)ₙC₁₋₆alkyl;
R² is H or C₁₋₆alkyl optionally substituted with one to six halogen; or
R¹ and R² together represent an oxo; or
R¹, R² and the atom(s) to which they are attached taken together form a 3- or 4-membered ring containing 0 or 1 heteroatom selected from NR^{f}, S, and O optionally substituted with one or two groups selected from F, CF₃ and CH₃;
R³ is H or C₁₋₆alkyl optionally substituted with one to six groups independently selected from —OR^{a} and halogen;
R^{a} and R^{b} are independently selected from H, C₁₋₁₀alkyl, C₂₋₁₀alkenyl, C₂₋₁₀alkynyl, Cy and Cy-C₁₋₁₀alkyl-, wherein said alkyl, alkenyl, alkynyl and Cy are optionally substituted with one to six substituents independently selected from halogen, amino, carboxy, C₁₋₄alkyl, OH, C₁₋₄alkoxy, aryl, heteroaryl, aryl-C₁₋₄alkyl-, hydroxy, CF₃, —OC(O)C₁₋₄alkyl, —OC(O)NRⁱR^{j}, and aryloxy; or
R^{a} and R^{b} together with the atom(s) to which they are attached form a heterocyclic ring of 4 to 7 members containing 0-2 additional heteroatoms independently selected from oxygen, sulfur and N—R^{f};
R^{c} is selected from C₁₋₆alkyl optionally substituted with one to six halogen, aryl and heteroaryl, wherein said aryl and heteroaryl are optionally substituted with halogen, —OC₁₋₆alkyl, C₁₋₆alkyl and wherein said alkyl is optionally substituted with one to six halogen;
R^{d} and R^{e} are independently H, halogen, aryl, heteroaryl, C₁₋₆alkyl or haloC₁₋₆alkyl;
R^{f} is selected from H, C₁₋₆alkyl, haloC₁₋₆alkyl, Cy, —C(O)C₁₋₆alkyl, —C(O)haloC₁₋₆alkyl, and —C(O)—Cy;
R^{g} is selected from (1) halogen, (2) CN, (3) C₁₋₆alkyl optionally substituted with one to eight groups independently selected from aryl, heteroaryl, halogen, NR^{a}R^{b}, C(O)R^{a}, C(OR^{a})R^{a}R^{b}, SR^{a} and OR^{a}, wherein aryl, heteroaryl and alkyl are each optionally substituted with one to six groups independently selected from halogen, CF₃, and COOH, (4) C₂₋₆alkenyl optionally substituted with one to six groups independently selected from halogen and OR^{a}, (5) Cy, (6) C(O)R^{a}, (7) C(O)OR^{a}, (8) CONR^{a}R^{b}, (9) OCONR^{a}R^{b}, (10) OR^{a}, (11) SH, (12) —S(O)ₙC₁₋₆alkyl, wherein alkyl is optionally substituted with one to six substituents selected from halogen, aryl, heteroaryl, OH, and OC(O)R^{a}, (13) —S(O)ₙaryl, (14) —S(O)ₙheteroaryl, (15) —NR^{a}S(O)ₙR^{b}, (16) —NR^{a}R^{b}, (17) —NR^{a}C(O)R^{b}, (18) —NR^{a}C(O)OR^{b}, (19) —NR^{a}C(O)NR^{a}R^{b}, (20) —S(O)ₙNR^{a}R^{b}, (21) NO₂, (22) C₅₋₈cycloalkenyl; wherein Cy is optionally substituted with one to eight groups independently selected from halogen, C(O)R^{a}, OR^{a}, C₁₋₃alkyl, aryl, heteroaryl and CF₃;
Rⁱ and R^{j} are independently selected from hydrogen, C₁₋₁₀alkyl, Cy and Cy-C₁₋₁₀alkyl-; or
Rⁱ and R^{j} together with the nitrogen atom to which they are attached form a ring of 5 to 7 members containing 0-2 additional heteroatoms independently selected from oxygen, sulfur and N—R^{f};
Cy is selected from heterocyclyl, aryl, and heteroaryl;
m is 1, 2 or 3; and
n is 0, 1 or 2.

The invention also encompasses pharmaceutical compositions containing a compound of formula I, and methods for treatment or prevention of prostaglandin mediated diseases using compounds of formula I.

The invention is described using the following definitions unless otherwise indicated.

The term “alkyl” refers to linear, branched and cyclic and bicyclic structures and combinations thereof, containing the indicated number of atoms. Non-limiting examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl4-propylnonyl, cyclopropyl, cyclobutyl, cyclopentyl, cyclohexyl, cycloheptyl, cyclopropylmethyl, cyclopentylethyl, methyl substituted cyclopropyl, ethyl substituted cyclobutyl, adamantyl, cyclododecylmethyl, 2-ethyl-1-bicyclo[4.4.0]decyl and the like. For example, the term C₁₋₆alkyl encompasses, in addition to acyclic alkyl groups having the indicated number of carbon atoms, —Cₓalkyl-C_{z}cycloalkyl wherein x is 0 to 3 and z is 3 to 6 with the proviso that x+z=3 to 6; similarly, C₁₋₁₀alkyl encompasses —Cₓalkyl-C_{z}cycloalkyl wherein x is 0 to 7 and z is 3 to 10 with the proviso that x+z=3 to 10.

“Alkoxy” means alkoxy groups of a straight, branched or cyclic configuration having the indicated number of carbon atoms. C₁₋₆alkoxy, for example, includes methoxy, ethoxy, propoxy, isopropoxy, and the like.

“Alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

“Aryl” means a 6-14 membered carbocyclic aromatic ring system comprising 1-3 benzene rings. If two or more aromatic rings are present, then the rings are fused together, so that adjacent rings share a common bond. Examples include phenyl and naphthyl.

“Cycloalkylidene” refers to the following bivalent radical where the points of attachement are on the same carbon atom:

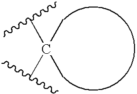

“Cycloalkylene” refers to the following bivalent radical where the points of attachment are on different carbon atoms:

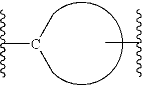

“Phenylene” refers to the following bivalent radical and includes 1,2-phenylene, 1,3-phenylene and 1,4-phenylene:

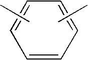

“Halogen” or “halo” includes F, Cl, Br, and I.

“Haloalkyl” means an alkyl group as described above wherein one or more hydrogen atoms have been replaced by halogen atoms, with up to complete substitution of all hydrogen atoms with halo groups. C₁₋₆haloalkyl, for example, includes —CF₃, —CH₂CF₃, —CF₂CF₃ and the like.

“Haloalkoxy” means an alkoxy group as described above in which one or more hydrogen atoms have been replaced by halogen atoms, with up to complete substitution of all hydrogen atoms with halo groups. C₁₋₆haloalkoxy, for example, includes —OCF₃, —OCH₂CF₃, —OCF₂CF₃ and the like.

“Heterocyclyl” refers to a non-aromatic ring having 1 to 4 heteroatoms said ring being isolated or fused to a second ring selected from 3- to 7-membered alicyclic ring containing 0 to 4 heteroatoms, aryl and heteroaryl, wherein said heteroatoms are independently selected from O, N and S. Non-limiting examples of heterocyclyl include oxetanyl, azetidinyl, pyrrolidinyl, tetrahydrofuranyl, tetrahydrothienyl, morpholinyl, piperzinyl, piperidinyl, benzodiazepinyl, tetrahydroquinolinyl, tetrahydroisoquinolinyl, 1,3-dithiacyclopentane, dihydrobenzofuran, and the like.

The term “heteroaryl” (Het) as used herein represents a 5-10 membered aromatic ring system containing one ring or two fused rings, 1-4 heteroatoms, selected from O, S and N. Het includes, but is not limited to, tetrazolyl, benzothienyl, quinolinyl, benzothiazolyl, furanyl, pyrimidinyl, purinyl, naphthyridinyl, imidazolyl, isoxazolyl, isothiazolyl, oxadiazolyl, oxazolyl, pyrazolyl, pyridyl, pyrrolyl, tetrazinyl, thiazolyl, thiadiazolyl, thienyl, triazinyl, triazolyl, 1H-pyrrole-2,5-dionyl, 2-pyrone, 4-pyrone, pyrrolopyridine, furopyridine and thienopyridine.

“Therapeutically effective amount” means that amount of a drug or pharmaceutical agent that will elicit the biological or medical response of a tissue, a system, animal or human that is being sought by a researcher, veterinarian, medical doctor or other clinician.

The term “treatment” or “treating” includes alleviating, ameliorating, relieving or otherwise reducing the signs and symptoms associated with a disease or disorder.

The term “prophylaxis” means preventing or delaying the onset or the progression of a disease or disorder, or the signs and symptoms associated with such disease or disorder.

The term “composition,” as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, and pharmaceutically acceptable excipients.

For compounds of formula I, examples of A include, but are not limited to, CH₂, CH₂CH₂, CH₂CH(CH₃), CH(Cl), CH₂CF₂CH₂, CH(Cl)CH₂CH(F), OCH₂, OCH₂CH₂, SCH₂ and SCH₂CH₂. Examples of Q′ include, but are not limited to, CO₂H, CONH₂, CONHCH₃, CONHPh, CON(CH₃)₂, CON(CH₂)₄, CONHSO₂CH₃, SO₂NHPh, tetrazolyl and the like.

Examples of X include, but are not limited to, CH₂, CH₂CH₂, CH₂CH(CH3), CH(Cl), CH(CH₃), CH(Ph), CH₂CH(CF₃), CF₂CH₂, SO₂, C(O), CH₂C(O), CH₂C(O)O, CH₂C(O)OCH₂, CH═CH, CH₂CH═CHCH₂, CH₂C≡C, 1,4-phenylene, 1,1-cyclopropylidene, 1,3-cyclohexylene, and the like.

Examples of Ar include, but are not limited to, phenyl, 2-, 3-, 4-chlorophenyl, 2-, 3-, 4-bromophenyl, 2-, 3-, 4-fluorophenyl, 3,4-diclorophenyl, 2,3-dichlorophenyl, 2,4-dichlorophenyl, 2,5-dichlorophenyl, 2,6-dichlorophenyl, 3,5-dichlorophenyl, 3-chloro-4-fluorophenyl, 2-chloro-4-fluoro-phenyl, 4-chloro-2-fluorophenyl, 2-cyanophenyl, 4-methylphenyl, 4-isopropylphenyl, 4-trifluoromethyl, biphenyl, naphthyl, 3-methoxyphenyl, 3-carboxyphenyl, 2-carboxamidophenyl, 4-methoxyphenyl, 3-phenoxyphenyl, 4-(4-pyridyl)phenyl, 4-methylsulfonylphenyl, 3-dimethylaminophenyl, 5-tetrazolyl, 1-methyl-5-tetrazolyl, 2-methyl-5-tetrazolyl, 2-benzothienyl, 2-benzofuranyl, 2-indolyl, 2quinolinyl, 7-quinolinyl, 2-benzothiazolyl, 2-benzimidazolyl, 1-benzotriazolyl, 2-furanyl, 3-furanyl, 2-imidazolyl, 5-imidazolyl, 5-isoxazolyl, 4-isoxazolyl, 4-isothiazolyl, 1,2,4-oxadiazol-5-yl, 2-oxazolyl, 4-oxazolyl, 4-pyrazolyl, 5-pyrazolyl, 2-pyridyl, 3-pyridyl, 2-pyrazinyl, 5-pyrimidinyl, 2-pyrrolyl, 4-thiazolyl, 1,2,4-thiadiazol-3-yl, 1,2,5-thiadiazol-4-yl, 1,2,3-thiadiazol-4-yl, 1,2,5-oxadiazol-4-yl, 1,2,3-oxadiazol-4-yl, 1,2,4-triazol-5-yl, 1,2,3-triazol-4-yl, 3-thienyl, 1,2,4-triazol-5-yl, pyrrolopyridine, furo[3,2-b]pyridin-2-yl, thieno[2,3-b]pyridin2-yl, 5(H)-2-oxo-4-furanyl, 5(H)-2-oxo-5-furanyl, (1H,4H)-5-oxo-1,2,4-triazol-3-yl, 4-oxo-2-benzopyranyl, and the like.

Examples of Z¹, Z², Z³ and Z⁴ include, but are not limited to, N, N→O, CH, C—CH₃, C—CH(CH₃)₂, C—Ph, C—Cl, C—Br, C—F, C—CF₃, C—C(O)CH₃, C—C(O)OH, C—C(O)NH₂, C—C(O)N(CH₂)₂O(CH₂)₂, C—OCH₃, C—OCF₃, C—OPh, C—SCH₃, C—SOCH₃, C—SO₂CH₃, C—SO₂Ph, C—NH₂, C—N(CH₃)₂, C—N(CH₃)C(O)CH₃, C—N(CH₃)C(O)OCH₃, C—NHC(O)NHCH₃, C—cyclopropyl, C—cyclobutyl, C—cyclopentyl, C—oxazolyl, C—thienyl, C—CH═CH₂, C—C(OH)(CH₃)₂, and the like.

Examples of R¹ include, but are not limited to, hydrogen, cyano, OH, CH₃, CH₂CH₃, CF₃, CH₂CH₂Cl, cyclopropyl, and the like.

Examples of R² include, but are not limited to, hydrogen, CH₃, CH₂CH₃, CF₃, CH₂CH₂Cl, cyclopropyl, and the like. R¹ and R² together may also represent oxo.

Examples of R³ include, but are not limited to, hydrogen, CH₃, CH₂CH₃, CF₃, CH₂CH₂Cl, CH₂CH₂OH, cyclopropyl, and the like.

In one embodiment of formula I, the moiety Q is CH₂CO₂H.

In a second embodiment of formula I are compounds wherein the moiety X—Ar is —(CR^{d}R^{e})ₐ-(CR^{d}R^{e})_{b}-aryl, —SO₂-aryl or —C(O)-aryl, wherein said aryl is naphthyl or phenyl optionally substituted with 1 to 2 groups selected from R^{g}. In one subset thereof, X—Ar is —(CR^{d}R^{e})ₐ—(CR^{d}R^{e})_{b}-aryl, —SO₂-aryl or —C(O)-aryl, wherein said aryl is naphthyl or phenyl optionally substituted with 1 to 2 groups selected from R^{g}. In another subset thereof, X—Ar is benzyl or α-methylbenzyl wherein the phenyl moiety is substituted with one to three chlorine atoms.

In a third embodiment of formula I are compounds wherein Z³ is nitrogen and Z¹, Z² and Z⁴ are independently selected from CH and CR^{g}. In one subset, one of Z¹, Z² and Z⁴ is CR^{g}, and the others are CH. In another subset, Z¹ is CR^{g}, Z² and Z⁴ are each CH. In another subset, Z¹ is C—SO₂—C₁₋₃alkyl, Z² and Z⁴ are each CH.

In a fourth embodiment of formula I are compounds wherein m is 1 or 2. In one subset, m is 1. ln a second subset m is 2.

In a fifth embodiment of formula I are compounds where R¹, R² and R³ are each hydrogen, or R¹ and R² together is oxo, and R³ is hydrogen.

One group of compounds within formula I is represented by the formula Ia: 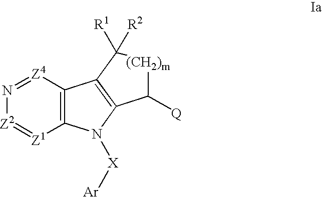
wherein Ar, Q, X, Z¹, Z², Z⁴, R¹, R² and m are as defined under formula I. In one embodiment of formula Ia are compounds where Q is CH₂CO₂H. In another embodiment X is CH₂, CH(CH₃), SO₂ or C(O), and in a subset thereof are compounds wherein X is CH₂; in another subset thereof are compounds wherein X is CH(CH₃). In a third embodiment are compounds wherein Ar is phenyl optionally substituted with one to three groups selected from R^{g}, and a subset thereof are compounds wherein R^{g} is halogen. In a fourth embodiment are compounds wherein Z² and Z⁴ are each CH.

Another group of compounds within formula I is represented by the formula Ib: 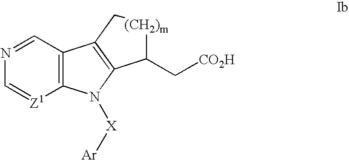
wherein Z¹ and m are as defined under formula I; Ar is phenyl optionally substituted with one or two R^{g} groups, and X is CH₂ or CH(CH₃). In one embodiment of formula Ib are compounds wherein m is 1 or 2. In a second embodiment are compounds where Z¹ is C—SO₂—C₁₋₃alkyl or C—SO₂NR^{a}R^{b}. In third embodiment are compounds wherein Ar is phenyl substituted with one or two halogen atoms, and a subset thereof are compounds wherein Ar is 4-chlorophenyl.

Compounds of Formula I of the present invention can be prepared according to the synthetic routes outlined in Schemes 1 to 6 and by following the methods described herein. Both the schemes and specific examples provided herein are for illustrative purpose, and a person skilled in the art will appreciate that other compounds of the present invention may be analogously prepared using the illustrative procedures, or they may be obtained from exemplified compounds via functional group interconversion, or they mnay be prepared by other procedures that are known to persons skilled in the art of organic synthesis.

Intermediate compounds of Formula IV may be prepared by the method presented in Scheme 1 from an appropriately substituted phenyl hydrazine (II). Reaction of II with an appropriate cycloalkanone III under Fisher Indole or similar conditions gives IV.¹

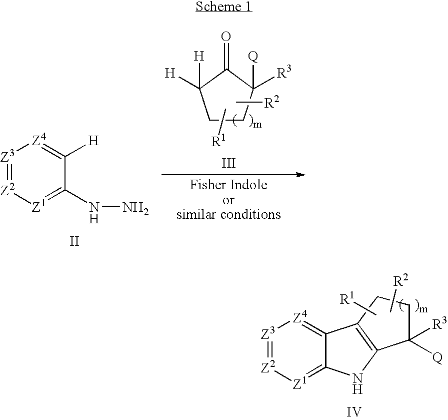

Compounds of Formula IV may alternatively be prepared by the method presented in Scheme 2 from an appropriately substituted arninopyridine (V). Reaction of V with iodine yields VI.^{2,3} Condensation with an appropriate cycloalkanone III followed by the cyclization under Heck or similar metal catalysis conditions leads to indole IV.⁴

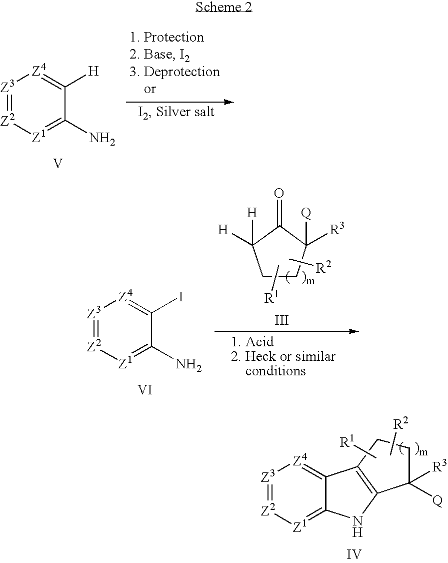

Compounds of Formula III may be prepared by the method presented in Scheme 3 from an appropriately substituted silyl enol ether (VII) or an appropriately substituted enamine (VI).

Addition of an appropriate electrophile such as QY (wherein Y represents a halogen or a leaving group) in the presence of a base such as an alkyl lithium or a Lewis acid such as silver trifluoroacetate with the silyl enol ether VII gives the cycloalkanone III.⁵⁶ The compound of formula III may alternatively be prepared from the addition of QY on an appropriately substituted enamine VIII under Stork Enarnine or similar conditions.⁷

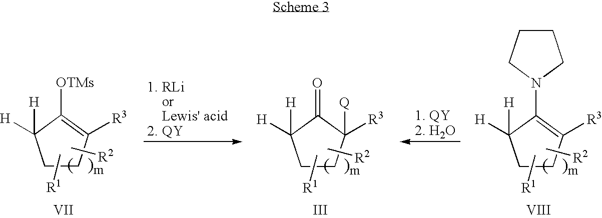

Intermediate compounds of Formula X may be prepared by the method presented in Scheme 4 from an appropriately substituted indole (IX). Bromination of IX may be accomplished with bromine in a polar and basic solvent such as pyridine followed by the reduction of excess reagents under acid conditions to generate the indole X.⁸

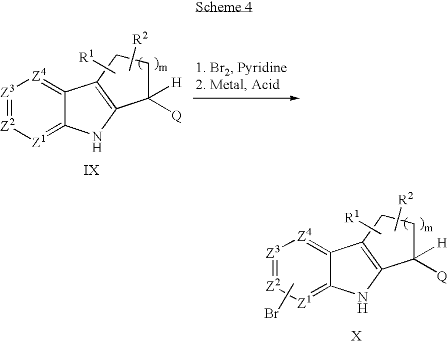

Compounds of Formula I may be prepared by the method presented in Scheme 5 from an appropriately substituted indole (IV). Alkylation of IV with the appropriate electrophile such as ArXY (wherein Y represents a halogen or a leaving group) in the presence of a base and in a suitable solvent such as DMF gives I.

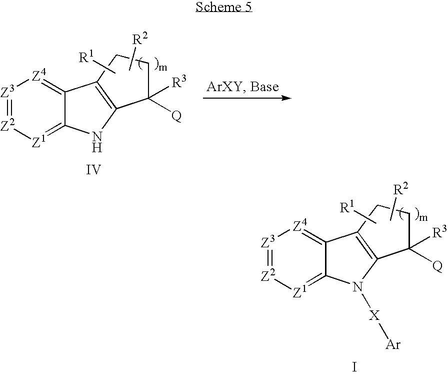

Compounds of Formula I may alternatively be prepared by the method presented in Scheme 6 from an appropriately substituted bromoindole (XI) from compound of formula X following the coupling reaction described in Scheme 5. Palladium coupling or similar reactions of bromoindole XI with an appropriate organometallic compound R³M (wherein M represents a metal such as B, Mg, Zn or Sn) leads to compound I.^{9,10} The same bromoindole XI may alternatively first react with a suitable metallation agent, such as n-BuLi, followed by trapping with an electrophile such as R³Y to give compound I.

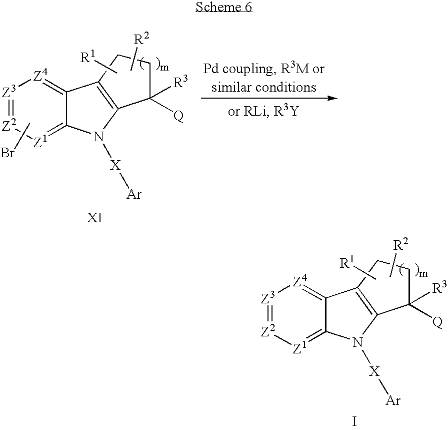

### REFERENCES

For illustrative examples, see:

1. Kelly, A. H.; Parrick, J. Can. J. Chem. 1966, 44, 2455-2459.
2. Estel, L.; Marsais, F.; Queguiner, G. J. Org. Chem. 1988, 53, 2740-2744.
3. Cooper, L. C.; Chicchi, G. G.; Dinnell, K.; Elliott, J. M.; Hollingworth, G. J.; Kurtz, M. M.; Locker, K. L.; Morrison, D.; Shaw, D. E.; Tsao, K.-L.; Watt, A. P.; Williams, A. R.; Swain, C. J.
4. Chen, C.-y.; Lieberman, D. R.; Larsen, R. D.; Verhoeven, T. R.; Reider, P. J. J. Org. Chem. 1997, 62, 2676-2677.
5. Hudlicky, T.; Short, R. P.; Revol, J.-M.; Ranu, B. C. J. Org. Chem. 1983, 48, 4453-4461.
6. Jefford, C. W.; Sledeski, A. W.; Lelandais, P.; Boukouvalas, J. Tetrahedron Lett. 1992, 33, 1855-1858.
7. Begley, M. J.; Cooper, K.; Pattenden, G. Tetrahedron 1981, 37, 4503-4508.
8. Unangst, P. C.; Connor, D. T.; Miller, S. R. J. Heterocyclic Chem. 1996, 33, 1627-1630.
9. Stille, J. K. Angew. Chem., Int. Ed. Engl. 1986, 25, 508-524.
10. Farina, V.; Krishnan, B. J. Am. Chem. Soc. 1991, 113, 9585-9595.
   Representative Compounds

Representative compounds of formula I are shown in the following Tables, the substituents are as indicated. Each entry is intended to include the racemic or diastereomeric mixture, and the individual enantiomers and/or diastereomers. Methods for the resolution of enantiomers and for the separation of diastereomers are well known to those skilled in the art; selective illustration of separation of diastereomers is also described in the Examples herein below.

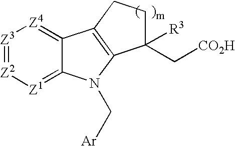

| Z¹ | Z² | Z³ | Z⁴ | R³ | Ar | m |
|---|---|---|---|---|---|---|
| N | CH | C—Br | CH | H | 4-Cl-phe | 1 |
| N | CH | C—COCH₃ | CH | H | 4-Cl-phe | 1 |
| N | CH | C—SO₂Me | CH | H | pyridin-3-yl | 1 |
| N | CH | C—SOMe | CH | H | 4-Cl-phe | 1 |
| N—O | CH | C—SOMe | CH | H | 4-Cl-phe | 1 |
| N | CH | C—SOMe | CH | H | 4-Cl-phe | 2 |
| N | CH | C—SOMe | CH | H | 4-Cl-phe | 3 |
| N | CH | CH | C—CO₂Me | H | 4-Cl-phe | 1 |
| C—SO₂Me | N | CH | CH | H | 4-Cl-phe | 1 |
| C—SO₂Me | N—O | CH | CH | H | 4-Cl-phe | 1 |
| C—SO₂Me | N | CH | CH | H | 4-Cl-phe | 2 |
| C—SO₂Me | N | CH | CH | H | 4-Cl-phe | 3 |
| C—SO₂Me | N | CH | CH | CH₃ | 4-Cl-phe | 1 |
| CH | N | C—Br | CH | H | 2,4-Cl₂-phe | 1 |
| CH | N | C—CH═CH₂ | CH | H | 4-Cl-phe | 1 |
| CH | N | C-cyclo-Pr | CH | H | thiophen-2-yl | 1 |
| CH | N | C-(thiophen-2-yl) | CH | H | 4-Cl-phe | 1 |
| C—CF₃ | N | CH | C-F | H | 2,4-Cl₂-phe | 1 |
| C—CF₃ | N | CH | CH | H | 3,4-Cl₂-phe | 1 |
| C—Br | CH | N | CH | H | 4-Cl-phe | 1 |
| C—SO₂NMe₂ | CH | N | CH | H | 4-Cl-phe | 1 |
| C—SO₂Me | CH | N | CH | H | 4-Cl-phe | 1 |
| C—SO₂Me | CH | N—O | CH | H | 4-Cl-phe | 1 |
| C—SO₂Me | CH | N | CH | H | 4-Cl-phe | 2 |
| C—SO₂Me | CH | N | CH | H | 4-Cl-phe | 3 |
| C—SO₂Me | CH | N | CH | CH₃ | 4-Cl-phe | 1 |
| C—SO₂Me | CH | N—O | CH | CH₃ | 4-Cl-phe | 1 |
| C—SO₂Me | CH | N | CH | CH₃ | 4-Cl-phe | 2 |
| C—SO₂Me | CH | N | CH | CH₃ | 4-Cl-phe | 3 |
| C—SO₂Et | CH | N | CH | H | 4-Cl-phe | 1 |
| C—SO₂Me | CH | N | CH | H | 4-CN-phe | 1 |
| C—SO₂Me | CH | N | CH | H | 2,4,6-Cl₃-phe | 1 |
| C—SO₂Me | CH | N | CH | H | 2,6-Cl₂-phe | 1 |
| C—SO₂Me | CH | N | CH | H | 7-Cl-quinolin- | 1 |
| | | | | | 2-yl | |
| C—SO₂Me | CH | N | CH | H | 4-CF₃-phe | 1 |
| C-(oxazol-2-yl) | CH | N | CH | H | 4-Cl-phe | 1 |
| C—Br | CH | CH | N | H | 4-Cl-phe | 1 |
| C—SO₂Me | CH | CH | N | H | 4-Cl-phe | 1 |
| C—SO₂Me | CH | CH | N—O | H | 4-Cl-phe | 1 |
| C—SO₂Me | CH | CH | N | H | 4-Cl-phe | 2 |
| C—SO₂Me | CH | CH | N | H | 4-Cl-phe | 3 |
| C—SO₂Me | CH | CH | N | CH₃ | 4-Cl-phe | 1 |
| C—SO₂Me | CH | CH | N—O | CH₃ | 4-Cl-phe | 1 |
| C—SO₂Me | CH | CH | N | CH₃ | 4-Cl-phe | 2 |
| C—SO₂Me | CH | CH | N—O | CH₃ | 4-Cl-phe | 2 |
| C—SO₂Me | CH | CH | N | CH₃ | 4-Cl-phe | 3 |
| C—SO₂Me | CH | C—CH(OH)CH₃ | N | H | 4-Cl-phe | 1 |
| C—SO₂Me | C—F | CH | N | H | 4-Cl-phe | 1 |
| C-i-Pr | C—F | CH | N | H | 4-Cl-phe | 1 |
| C—CF₃ | CH | CH | N | H | 4-Cl-phe | 1 |
| C—C(OH)Me₂ | CH | CH | N | H | 4-Cl-phe | 1 |
| C—CF3 | CH | CH | N | H | thiophen-2-yl | 1 |

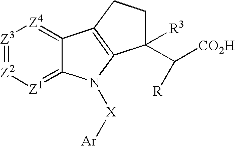

| Z¹ | Z² | Z³ | Z⁴ | R | R³ | X | Ar |
|---|---|---|---|---|---|---|---|
| C—SO₂Me | N | C—CH(OH)Me | CH | CH₃ | H | CH₂ | 4-Cl-phe |
| C—SO₂Me | CH | N | CH | CH₃ | H | CH₂ | 4-Cl-phe |
| N | C—CH₃ | CH | CH | H | H | SO₂ | 2,4-Cl₂-phe |
| N | CH | CH | C-cBu | H | H | SO₂ | 3,4-Cl₂-phe |
| N | CH | CH | C-cBu | H | H | SO₂ | 4-Cl-phe |
| N | CH | CH | CH | H | CH₃ | SO₂ | 4-Cl-phe |
| C—CF₃ | CH | CH | N | H | H | 1,1-cPr | 4-Cl-phe |
| C—Br | CH | N | CH | H | H | CHCH₃ | 4-Cl-phe |
| C—SO₂Me | CH | N | CH | H | H | CHCH₃ | 4-Cl-phe |
| C—SO₂Me | CH | N | CH | H | H | (CH₂)₂ | 4-Cl-phe |
| C—F | CH | N | CH | H | H | CO | 2,4-Cl₂-phe |
| C—F | CH | N | CH | H | H | CO | 3,4-Cl₂-phe |
| C—F | CH | N | CH | H | H | CO | 4-Cl-phe |
| C—CF₃ | CH | N | CH | H | H | CHCH₃ | 3,4-Cl₂-phe |

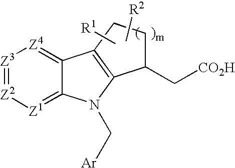

| Z¹ | Z² | Z³ | Z⁴ | R¹ | R² | Ar | m |
|---|---|---|---|---|---|---|---|
| N | CH | C—COCH₃ | CH | 1-OH | H | 4-Cl-phe | 1 |
| N—O | CH | C—COCH₃ | CH | 1-OH | H | 4-Cl-phe | 1 |
| N | CH | C—COCH₃ | CH | 1-OH | H | 4-Cl-phe | 2 |
| N | CH | C—COCH₃ | CH | 1-OH | H | 4-Cl-phe | 3 |
| C—CF₃ | N | CH | C—F | 1-oxo | H | 2,4-Cl₂-phe | 1 |
| C—CF₃ | N—O | CH | C—F | 1-oxo | H | 2,4-Cl₂-phe | 1 |
| C—CF₃ | N | CH | C—F | 1-oxo | H | 2,4-Cl₂-phe | 2 |
| C—CF₃ | N—O | CH | C—F | 1-oxo | H | 2,4-Cl₂-phe | 2 |
| C—CF₃ | N | CH | C—F | 1-oxo | H | 2,4-Cl₂-phe | 3 |
| C—CF₃ | CH | N | C—H | 1-oxo | 2-CH₃ | 2,4-Cl₂-phe | 1 |
| C—CF₃ | CH | N | C—H | 1-oxo | H | 2,4-Cl₂-phe | 1 |
| C—CF₃ | CH | N—O | C—H | 1-oxo | H | 2,4-Cl₂-phe | 1 |
| C—CF₃ | CH | N | C—H | 1-oxo | H | 2,4-Cl₂-phe | 2 |
| C—CF₃ | CH | N—O | C—H | 1-oxo | H | 2,4-Cl₂-phe | 2 |
| C—CF₃ | CH | N | C—H | 1-oxo | H | 2,4-Cl₂-phe | 3 |
| C—CF₃ | CH | N—O | C—H | 1-oxo | H | 2,4-Cl₂-phe | 3 |
| C—SO₂Me | CH | N | C—H | 1-CH₃ | 1-CH₃ | 2,4-Cl₂-phe | 1 |
| C—SO₂Me | CH | N | CH | H | 2-OH | 4-Cl-phe | 1 |
| C—SO₂Me | CH | N | CH | 1-OH | H | 4-Cl-phe | 1 |
| C—CF₃ | CH | CH | N | 1-OH | H | thiophen-2-yl | 1 |

Optical Isomers—Diastereomers—Tautomers

Compounds of formula I contain one or more asymmetric centers and can thus occur as racemates and racemic mixtures, single enantiomers, diastereomeric mixtures and individual diastereomers. The present invention is meant to comprehend all such isomeric forms of the compounds of formula I.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of formula I.

Compounds of formula I may be separated into diastereomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid or base as a resolving agent, or by chiral separation techniques such as separation by HPLC using a chiral column.

Alternatively, any enantiomer of a compound of the general formula I or Ia may be obtained by stereospecific synthesis using optically pure starting materials or reagents of known configuration.

Salts

The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethyl-aminoethanol, 2-dimethylaminoethanol, ethanolamine, ethylenediamine, N-ethyl-morpholine, N-ethyl-piperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromnic, hydrochloric, maleic, phosphoric, sulfuric, and tartaric acids.

It will be understood that, unless otherwise specified, references to the compound of formula I are meant to also include the pharmaceutically acceptable salts.

Utilities

Compounds of formula I are antagonists of prostaglandin D2. The ability of compounds of formula I to interact with prostaglandin D2 receptor makes them useful for preventing or reversing undesirable symptoms caused by prostaglandins in a mammalian, especially human subject. The antagonism of the actions of prostaglandin D2 indicates that the compounds and pharmaceutical compositions thereof are useful to treat, prevent, or ameliorate in mammals and especially in humans: respiratory conditions, allergic conditions, pain, inflammatory conditions, mucus secretion disorders, bone disorders, sleep disorders, fertility disorders, blood coagulation disorders, trouble of the vision as well as immune and autoimmune diseases. In addition, such a compound may inhibit cellular neoplastic transformations and metastic tumor growth and hence can be used in the treatment of cancer. Compounds of formula I may also be of use in the treatment and/or prevention prostaglandin D2 mediated proliferation disorders such as may occur in diabetic retinopathy and tumor angiogenesis. Compounds of formula I may also inhibit prostanoid-induced smooth muscle contraction by antagonizing contractile prostanoids or nimicking relaxing prostanoids and hence may be use in the treatment of dysmenorrhea, premature labor and eosinophil related disorders.

Accordingly, another aspect of the invention provides a method of treating or preventing a prostaglandin D2 mediated disease comprising administering to a mammalian patient in need of such treatment a compound of formula I in an amount which is effective for treating or preventing said prostaglandin D2 mediated disease. Prostaglandin D2 mediated diseases include, but are not limited to, allergic rhinitis, nasal congestion, rhinorrhea, perennial rhinitis, nasal inflammation, asthma including allergic asthma, chronic obstructive pulmonary diseases and other forms of lung inflammation; pulmonary hypotension; sleep disorders and sleep-wake cycle disorders; prostanoid-induced smooth muscle contraction associated with dysmenorrhea and premature labor; eosinophil related disorders; thrombosis; glaucoma and vision disorders; occlusive vascular diseases, such as for example atherosclerosis; congestive heart failure; diseases or conditions requiring a treatment of anti-coagulation such as post-injury or post surgery treatment; rheumatoid arthritis and other inflammatory diseases; gangrene; Raynaud's disease; mucus secretion disorders including cytoprotection; pain and migraine; diseases requiring control of bone formation and resorption such as for example osteoporosis; shock; thermal regulation including fever; rejection in organ transplant and by-pass surgery, and immune disorders or conditions in which immunoregulation is desirable. Compounds of formula I may further be used in combination with nicotinic acid or a salt or solvate thereof, or another nicotinic acid receptor agonist for treating atherosclerosis in a human in the absence of substantial flushing. More particularly the disease and/or conditions to be treated is one mediated by prostaglandin D2 such as nasal congestion, allergic rhinitis, pulmonary congestion, and asthma including allergic asthma, as well as flushing induced by niacin.

Dose Ranges

The magnitude of prophylactic or therapeutic dose of a compound of formula I will, of course, vary with the nature and the severity of the condition to be treated and with the particular compound of formula I and its route of administration. It will also vary according to a variety of factors including the age, weight, general health, sex, diet, time of administration, rate of excretion, drug combination and response of the individual patient. In general, the daily dose is from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for the oral administration of humans may contain from 0.05 mg to 5 g of active agent compounded with an appropriate and convenient amount of carrier material which may vary from about 5 to about 99.95 percent of the total composition. Dosage unit forms will generally contain between from about 0.1 mg to about 0.4 g of an active ingredient, typically 0.5 mg, 1 mg, 2 mg, 5 mg, 10 mg, 25 mg, 50 mg, 100 mg, 200 mg, or 400 mg.

Pharmaceutical Compositions

Another aspect of the present invention provides pharmaceutical compositions comprising a compound of formula I with a pharmaceutically acceptable carrier. The term “composition,” as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

For the treatment of any of the prostanoid mediated diseases compounds of formula I may be administered orally, by inhalation spray, topically, parenterally or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of formula I may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing the compound of formula I are employed. (For purposes of this application, topical application shall include mouth washes and gargles.) Topical formulations may generally be comprised of a pharmaceutical carrier, cosolvent, emulsifier, penetration enhancer, preservative system, and emollient.

Combinations with Other Drugs

For the treatment and prevention of prostaglandin mediated diseases, compound of formula I may be co-administered with other therapeutic agents. Thus in another aspect the present invention provides pharmaceutical compositions for treating prostaglandin D2 mediated diseases comprising a therapeutically effective amount of a compound of formula I and one or more other therapeutic agents. Suitable therapeutic agents for combination therapy with a compound of formula I include: (1) a prostaglandin receptor antagonist; (2) a corticosteroid such as triamcinolone acetonide; (3) a β-agonist such as salmeterol, formoterol, terbutaline, metaproterenol, albuterol and the like; (4) a leukotriene modifier, such as a leukotriene antagonist or a lipooxygenase inhibitor such as montelukast, zafirlukast, pranlukast, or zileuton; (5) an antihistamine (histamine H1 antagonist) such as bromophenirarnine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine, pyrilamine, astemizole, norastemizole, terfenadine, loratadine, cetirizine, levocetirizine, fexofenadine, desloratadine, and the like; (6) a decongestant including phenylephrine, phenylpropanolamine, pseudoephedrine, oxymetazoline, epinephrine, naphazoline, xylo-metazoline, propylhexedrine, or levo-desoxyephedrine; (7) an antitussive including codeine, hydrocodone, caramiphen, carbetapentane, or dextromethorphan; (8) another prostaglandin ligand including prostaglandin F agonist such as latanoprost; misoprostol, enprostil, rioprostil, ornoprostol or rosaprostol; (9) a diuretic; (10) non-steroidal antiinflannnatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid. derivatives (diflunisal and flufenisal), oxicams (isoxicam, piroxicam, sudoxicam and tenoxicam), salicylates (acetyl salicylic acid, sulfasalazine) and the pyrazolones (apazone, bezpiperylon, feprazone, mofebutazone, oxyphenbutazone, phenylbutazone); (11) cyclooxygenase-2 (COX-2) inhibitors such as celecoxib and rofecoxib, etoricoxib and valdecoxib; (12) inhibitors of phosphodiesterase type IV (PDE-IV) e.g. Ariflo, roflumilast; (13) antagonists of the chemokine receptors, especially CCR-1, CCR-2, and CCR-3; (14) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrate, fenofibrate and bezafibrate), and probucol; (15) anti-diabetic agents such as insulin, sulfonylureas, biguanides (metforrnin), α-glucosidase inhibitors (acarbose) and glitazones (troglitazone, pioglitazone, englitazone, rosiglitazone and the like); (16) preparations of interferon beta (interferon beta-1a, interferon beta-1b); (17) anticholinergic agents such as muscarinic antagonists (ipratropium bromide and tiotropium bromide), as well as selective muscarinic M3 antagonists; (18) steroids such as beclomethasone, methylprednisolone, betamethasone, prednisone, dexamethasone, and hydrocortisone; (19) triptans commonly used for the treatment of migraine such as sumatriptan and rizatriptan; (20) alendronate and other treatments for osteoporosis; (21) other compounds such as 5-aminosalicylic acid and prodrugs thereof, antimetabolites such as azathioprine and 6-mercaptopurine, cytotoxic cancer chemotherapeutic agents, bradykinin (BK2 or BK1) antagonists, TP receptor antagonists such as seratrodast, neurokinin antagonists (NK1/NK2), VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206.

In addition, the invention encompasses a method of treating prostaglandin D₂ mediated diseases comprising: administering to a patient in need of such treatment a therapeutically effective amount of the compound of formula I, co-administered with one or more of such ingredients as listed immediately above. The amounts of active ingredients may be those commonly used for each active ingredient when it is administered alone, or in some instances the combination of active ingredients may result in lower dosage for one or more of the active ingredients.

Assays for Determining Biolocical Activity

Compounds of formula I can be tested using the following assays to determine their prostanoid antagonist or agonist activity in vitro and in vivo and their selectivity. The prostaglandin receptor activities demonstrated are DP, EP₁, EP₂, EP₃, EP₄, FP, IP and TP.

Stable Expression of Prostanoid Receptors in the Human Embryonic Kidney (HEK) 293(ebna) Cell Line

Prostanoid receptor cDNAs corresponding to full length coding sequences are subcloned into the appropriate sites of mammaian expression vectors and transfected into HEK 293(ebna) cells. HEK 293(ebna) cells expressing the individual cDNAs are grown under selection and individual colonies are isolated after 2-3 weeks of growth using the cloning ring method and subsequently expanded into clonal cell lines.

Prostanoid Receptor Binding Assays

HEK 293(ebna) cells are maintained in culture, harvested and membranes are prepared by differential centrifugation, following lysis of the cells in the presence of protease inhibitors, for use in receptor binding assays. Prostanoid receptor binding assays are performed in 10 mM MES/KOH (pH 6.0) (EPs, FP and TP) or 10 mM HEPES/KOH (pH 7.4) (DP and IP), containing 1 mM EDTA, 10 mM divalent cation and the appropriate radioligand. The reaction is initiated by addition of membrane protein. Ligands are added in dimethylsulfoxide which is kept constant at 1% (v/v) in all incubations. Non-specific binding is determined in the presence of 1 μM of the corresponding non-radioactive prostanoid. Incubations are conducted for 60 min at room temperature or 30° C. and terminated by rapid filtration. Specific binding is calculated by subtracting non specific binding from total binding. The residual specific binding at each ligand concentration is calculated and expressed as a function of ligand concentration in order to construct sigmoidal concentration-response curves for determination of ligand affinity.

Prostanoid Receptor Agonist and Antagonist Assays

Whole cell second messenger assays measuring stimulation (EP₂, EP₄, DP and IP in HBEK 293(ebna) cells) or inhibition (EP₃ in human erythroleukemia (HEL) cells) of intracellular cAMP accumulation or mobilization of intracellular calcium (EP₁, FP and TP in HEK 293(ebna) cells stably transfected with apo-aequorin) are performed to determine whether receptor ligands are agonists or antagonists. For cAMP assays, cells are harvested and resuspended in HBSS containing 25 mnM HEPES, pH 7.4. Incubations contain 100 μM RO-20174 (phosphodiesterase type IV inhibitor, available from Biomol) and, in the case of the EP₃ inhibition assay only, 15 μM forskolin to stimulate cAMP production. Samples are incubated at 37° C. for 10 min, the reaction is terminated and cAMP levels are then measured. For calcium mobilization assays, cells are charged with the co-factors reduced glutathione and coelenterazine, harvested and resuspended in Ham's F12 medium. Calcium mobilization is measured by monitoring luminescence provoked by calcium binding to the intracellular photoprotein aequorin. Ligands are added in dimethylsulfoxide which is kept constant at 1% (v/v) in all incubations. For agonists, second messenger responses are expressed as a function of ligand concentration and both EC₅₀ values and the maximum response as compared to a prostanoid standard are calculated. For antagonists, the ability of a ligand to inhibit an agonist response is determined by Schild analysis and both K_{B} and slope values are calculated.

Prevention of PGD2 or Allergen Induced Nasal Congestion in Allergic Sheep

Animal preparation: Healthy adult sheeps (18-50 kg) are used. These animals are selected on the basis of a natural positive skin reaction to an intradermal injection of Ascaris suum extract.

Measurements of nasal congestion: The experiment is performed on conscious animals. They are restrained in a cart in a prone position with their heads immobilized. Nasal airway resistance (NAR) is measured using a modified mask rhinometry technique. A topical anesthesia (2% lidocaine) is applied to the nasal passage for the insertion of a nasotracheal tube. The maximal end of the tube is connected to a pneumotachograph and a flow and pressure signal is recorded on an oscilloscope linked to a computer for on-line calculation of NAR. Nasal provocation is performed by the administration of an aerosolized solution (10 puffs/nostril). Changes in the NAR congestion are recorded prior to and for 60-120 minutes post-challenge.

Prevention of PGD2 and Allergen Induced Nasal Obstruction in Cynomolgus Monkey

Animal preparation: Healthy adult male cynomolgus monkeys (4-10 kg) are used. These animals are selected on the basis of a natural positive skin reaction to an intradermal injection of Ascaris suum extract. Before each experiment, the monkey selected for a study is fasted overnight with water provided at libitum. The next morning, the animal is sedated with ketamine (10-15 mg/kg i.m.) before being removed from its home cage. It is placed on a heated table (36° C.) and injected with a bolus dose (5-12 mg/kg i.v.) of propofol. The animal is intubated with a cuffed endotracheal tube (4-6 mm I.D.) and anesthesia is maintained via a continuous intravenous infusion of propofol (25-30 mg/kg/h). Vital signs (heart rate, blood pressure, respiratory rate, body temperature) are monitored throughout the experiment.

Measurements of nasal congestion: A measurement of the animal respiratory resistance is taken via a pneumotachograph connected to the endotracheal tube to ensure that it is normal. An Ecovision accoustic rhinometer is used to evaluate nasal congestion. This technique gives a non-invasive 2D echogram of the inside of the nose. The nasal volume and the minimal cross-sectional area along the length of the nasal cavity are computed within 10 seconds by a laptop computer equipped with a custom software (Hood Laboratories, Mass, U.S.A.). Nasal challenge is delivered directly to the animal's nasal cavity (50 μL volume). The changes in nasal congestion are recorded prior to and for 60-120 minutes post-challenge. If nasal congestion occurs, it will translate into a reduction in the nasal volume.

Pulmonary Mechanics in Trained Conscious Squirrel Monkeys

The test procedure involves placing trained squirrel monkeys in chairs in aerosol exposure chambers. For control purposes, pulmonary mechanics measurements of respiratory parameters are recorded for a period of about 30 minutes to establish each monkey's normal control values for that day. For oral administration, compounds are dissolved or suspended in a 1% methocel solution (methylcellulose, 65 HG, 400 cps) and given in a volume of 1 mL/kg body weight. For aerosol administration of compounds, a DeVilbiss ultrasonic nebulizer is utilized. Pretreatment periods vary from 5 minutes to 4 hours before the monkeys are challenged with aerosol doses of either PGD2 or Ascaris suum antigen; 1:25 dilution.

Following challenge, each minute of data is calculated by computer as a percent change from control values for each respiratory parameter including airway resistance (R_{L}) and dynamic compliance (C_{dyn}). The results for each test compound are subsequently obtained for a minimum period of 60 minutes post challenge which are then compared to previously obtained historical baseline control values for that monkey. In addition, the overall values for 60 minutes post-challenge for each monkey (historical baseline values and test values) are averaged separately and are used to calculate the overall percent inhibition of mediator or Ascaris antigen response by the test compound. For statistical analysis, paired t-test is used. (References: McFarlane, C. S. et al., Prostaglandins, 28, 173-182 (1984) and McFarlane, C. S. et al., Agents Actions, 22, 63-68 (1987).)

Prevention of Induced Bronchoconstriction in Allergic Sheep

Animal Preparation: Adult sheep with a mean weight of 35 kg (range, 18 to 50 kg) are used. All animals used meet two criteria: a) they have a natural cutaneous reaction to 1:1,000 or 1:10,000 dilutions of Ascaris suum extract (Greer Diagnostics, Lenois, N.C.); and b) they have previously responded to inhalation challenge with Ascaris suum with both an acute bronchoconstriction and a late bronchial obstruction (W. M. Abraham et al., Am. Rev. Resp. Dis., 128, 839-44 (1983)).

Measurement of Airway Mechanics: The unsedated sheep are restrained in a cart in the prone position with their heads immobilized. After topical anesthesia of the nasal passages with 2% lidocaine solution, a balloon catheter is advanced through one nostril into the lower esophagus. The animals are then intubated with a cuffed endotracheal tube through the other nostril using a flexible fiberoptic bronchoscope as a guide. Pleural pressure is estimated with the esophageal balloon catheter (filled with one mL of air), which is positioned such that inspiration produces a negative pressure deflection with clearly discernible cardiogenic oscillations. Lateral pressure in the trachea is measured with a sidehole catheter (inner dimension, 2.5 mm) advanced through and positioned distal to the tip of the nasotracheal tube. Transpulmonary pressure, the difference between tracheal pressure and pleural pressure, is measured with a differential pressure transducer (DP45; Validyne Corp., Northridge, Calif.). For the measurement of pulmonary resistance (R_{L}), the maximal end of the nasotrachel tube is connected to a pneumotachograph (Fleisch, Dyna Sciences, Blue Bell, Pa.). The signals of flow and transpulmonary pressure are recorded on an oscilloscope (Model DR-12; Electronics for Medicine, White Plains, N.Y.) which is linked to a PDP-11 Digital computer (Digital Equipment Corp., Maynard, Mass.) for on-line calculation of R_{L} from transpulmonary pressure, respiratory volume obtained by integration and flow. Analysis of 10-15 breaths is used for the determination of R_{L}. Thoracic gas volume (V_{tg}) is measured in a body plethysmograph, to obtain specific pulmonary resistance (SR_{L}=R_{L}·V_{tg}).

The invention will now be illustrated in the following non-limiting Examples in which, unless otherwise stated:
1. All the end products of the formula I were analyzed by NMR, TLC and elementary analysis or mass spectroscopy.
2. Intermediates were analyzed by NMR and TLC.
3. Most compounds were purified by flash chromatography on silica gel, recrystallization and/or swish (suspension in a solvent followed by filtration of the solid).
4. The course of reactions was followed by thin layer chromatography (TLC) and reaction times are given for illustration only.

The following intermediates were prepared according to literature procedures or purchased from the following vendor:
1. Ethyl 2-(2-oxocyclopentyl)acetate: Acros/Fisher Scientific.
2. (1R)-1-(4-Chlorophenyl)ethanol: Mathre, D. J.; Thompson, A. S.; Douglas, A. W.; Hoogsteen, K.; Carroll, J. D.; Corley, E. G.; Grabowski, E. J. J. J.Org. Chem. 1993, 58, 2880-2888.

### EXAMPLE 1

### (+/−)-[5-(4-Chlorobenzyl)-4-(methylsulfonyl)-5,6,7,8-tetrahydrocyclopenta[4,5]pyrrolo[3,2-c]pyridin-6-yl]acetic acid

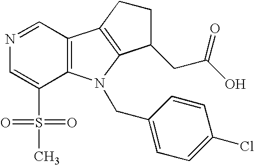

### Step 1. 2,2-Dimethyl-N-pyridin-4-ylpropanamide

To a 1.1 M solution of 4-aminopyridine in CH₂Cl₂ at −78° C. was added 1.3 equiv of Et₃N followed by 1.1 equiv of a 5.9 M solution of trimethylacetyl chloride in CH₂Cl₂. After allowing the reaction to warm to room temperature overnight, this mixture was poured into a separatory funnel containing brine/CH₂Cl₂. The layers were separated and the aqueous layer was extracted several times with CH₂Cl₂. The combined organic layer was dried over anhydrous Na₂SO₄, filtered and concentrated. The crude material was further purified by a pad of silica gel by eluting with a gradient from 60% EtOAc/hexanes to 100% EtOAc and the product was swished in hexanes twice to yield the title compound as an off-white solid. ¹H NMR (acetone-d₆) δ 8.95 (1H, br s), 8.42 (2H, dd), 7.69 (2H, dd), 1.31 (9H, s).

### Step 2. 4-Amino-3-iodopyridine

To a 0.5 M solution of 2,2-dimethyl-N-pyridinylpropanamide in THF at −78° C. was added 2.2 equiv of n-BuLi [1.7M] over 5-10 min. After allowing the reaction to warm up to −25° C. to −20° C. and stirring at this temperature for 1 h, this mixture was cooled again to −78° C. and treated with a 3.4 M solution of iodine in THF over 5 min. The reaction mixture was warmed to room temperature and poured into a separatory funnel containing aqueous Na₂S₂O₃/EtOAc. The layers were separated and the aqueous layer was extracted again with EtOAc. The combined organic layer was washed with Na₂S₂O₃, brine, and was then dried over anhydrous Na₂SO₄, filtered and concentrated. A 0.17 M solution of the resulting crude material in 10% aqueous H₂SO₄ (v/v) was then refluxed overnight. After allowing the reaction to cool to room temperature, aqueous 5N NaOH was added until the solution was slightly basic as indicated by pH paper. The aqueous layer was extracted twice with EtOAc. The combined organic layer was washed with brine, dried over anhydrous Na₂SO₄, filtered and concentrated. The crude material was further purified by a pad of silica gel by eluting with a gradient from 50% EtOAc/hexanes to 100% EtOAc and the product was swished in Et₂O/hexanes to yield the title compound as an off-white solid. ¹H NMR (acetone-d₆) δ 8.48 (1H, s), 8.02 (1H, d), 6.75 (1H, d) 5.75 (2H, br s).

### Step 3. (+/−)-Ethyl 5,6,7,8-tetrahydrocyclopenta[4,5]pyrrolo[3,2-c]pyridin-6-ylacetate

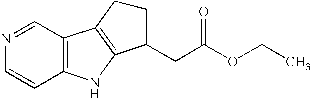

To a 4.6 M solution of 4-amino-3-iodopyridine in DMF was added 0.05 equiv of PTSA. The solution was degassed by bubbling with N₂ and 1.5 equiv of tetraethoxysilane was added, followed by 1.9 equiv of ethyl 2-(2-oxocyclopentyl)acetate. The final mixture was heated in a sealed tube to 250° C. for 600 sec in a microwave heating system (Smith Creator microwave oven set to 150 W power). The crude black gum was directly purified by flash chromatography on silica gel, eluting with a gradient from 100% hexanes to 50% EtOAc/hexanes. A 0.77 M solution of the resulting semi-pure brown oil in DMP was treated with 0.05 equiv of Pd(OAc)₂. The catalyst was dissolved in the reaction mixture with the help of ultrasound for few mnin. The solution was degassed by bubbling with N₂ and 2.8 equiv of Hunig's base was added. The final mixture was heated in a sealed tube to 150° C. for 300 sec under microwave heating system (Smith Creator microwave oven set to 150 W power). The reaction mixture was cooled to room temperature and poured into a separatory funnel containing diluted aqueous NH₄Cl/EtOAc. The layers were separated and the aqueous layer was extracted several times with EtOAc. The combined organic layer was washed with brine, dried over anhydrous Na₂SO₄, filtered and concentrated. A mixture of EtOAc/hexanes was added and the organic phase was filtered through Celite and concentrated. The crude material was further purified by crystallization at 0° C. with hexanes containing several drops of CH₂Cl₂ to yield the title compound as a pale brown solid. The structure of the title compound has been confnmed by 2D-COSY, 2D-HMQC and 2D-HMBC NMR experiments. ¹H NMR (acetone-d₆) δ 10.13 (1H, br s), 8.67 (1H, s), 8.14 (1H, d), 7.34 (1H, dd), 4.17 (2H, q), 3.62 (1H, m), 2.95-2.75 (3H, m), 2.69 (2H, m), 2.22 (1H, m), 1.24 (3H, t).

### Step 4. (+/−)-Ethyl (4-bromo-5,6,7,8-tetrahydrocyclopenta[4,5]pyrrolo[3,2-c]pyridin-6-yl)acetate

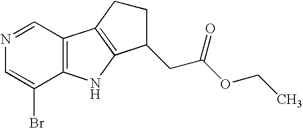

To a 0.14 M solution of ethyl 5,6,7,8-tetrahydrocyclopenta[4,5]pyrrolo[3,2-c]pyridin-6-ylacetate in CH₂Cl₂ at −78° C. was added 5.4 equiv of pyridine followed by 4.7 equiv of a 1.0 M solution of Br₂ in CH₂Cl₂. The mixture was stirred at −20° C. for 1 h and cooled to −78° C. A suspension of 6.5 equiv of Zn and 4.7 equiv of a 0.74 M solution of AcOH in THF was added portionwise. The resulting mixture was warmed to 0° C. and kept at this temperature for 15 min. The mixture was poured into aqueous saturated NaHCO₃ and extracted twice with EtOAc. The combined organic layer was washed with brine, dried over Na₂SO₄ and filtered through a very small pad of silica gel by eluting with 100% EtOAc, and was then concentrated. The crude material was further purified by crystallization at 0° C. with hexanes containing several drops of CH₂Cl₂ to yield the title compound as an off-white solid. The structure of the title compound has been confirmed by 2D-COSY, 2D-HMQC and 2D-HMBC NMR experiments. ¹H NMR (acetone-d₆) δ 10.33 (1H, br s), 8.64 (1H, s), 8.26 (1H, s), 4.17 (2H, q), 3.67 (1H, m), 2.95-2.80 (4H, m), 2.61 (1H, dd), 2.28 (1H, m), 1.24 (3H, t).

### Step 5. (+/−)-Ethyl [4-(methylsulfonyl)-5,6,7,8-tetrahydrocyclopenta[4,5]pyrrolo[3,2-c]pyridin-6-yl]acetate

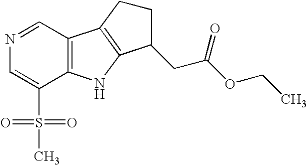

To a 0.11 M solution of ethyl (4-bromo-5,6,7,8-tetrahydrocyclopenta[4,5]pyrrolo[3,2-c]-pyridin-6-yl)acetate in DMSO was added 5.1 equiv of sodium methanesulphinate and 5.0 equiv of CuI. The suspension was degassed by bubbling with N₂ for 5 min and it was heated at 100° C. for 20 h under vigorous stiring. After allowing the reaction to cool to room temperature, this mixture was poured into a separatory funnel containing NH₄Cl/EtOAc. The layers were separated and the aqueous layer was extracted twice with EtOAc. The combined organic layer was dried over anhydrous Na₂SO₄, filtered through a pad of Celite and concentrated. The crude material was further purified by flash chromatography on silica gel eluting with a gradient from 100% hexanes to 100% EtOAc and the product was swished at 0° C. with hexanes containing several drops of CH₂Cl₂ to yield the title compound as a white solid. ¹H NMR (acetone-d₆) δ 10.49 (1H, br s), 8.91 (1H, s), 8.60 (1H, s), 4.18 (2H, q), 3.67 (1H, m), 3.30 (3H, s), 3.00-2.80 (4H, m), 2.70 (1H, dd), 2.27 (1H, m), 1.26 (3H, s

### Step 6. (+/−)-Ethyl [5-(4-chlorobenzyl)-4-(methylsulfonyl)-5,6,7,8-tetrahydrocyclopenta[4,5]pyrrolo[3,2-c]pyridin-6-yl]acetate

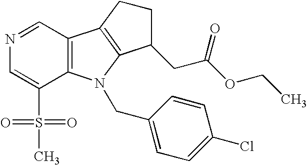

To a 0.16 M solution of ethyl [4-(methylsulfonyl)-5,6,7,8-tetrahydrocyclopenta[4,5]pyrrolo[3,2-c]pyridin-6-yl]acetate in THF was added 1.55 equiv of triphenylphosphine and 1.62 equiv of 4-chlorobenzyl alcohol followed by 1.52 equiv of a 0.48 M solution of di-tert-butyl azodicarboxylate in THF at room temperature. After 2 h of stirring, this mixture was directly purified by flash chromatography eluting with a gradient from 100% hexanes to 100% EtOAc to provide the title compound as a colorless oil. ¹H NMR (acetone-d₆) δ 8.99 (1H, s), 8.79 (1H, s), 7.37 (1H, d), 6.96 (1H, d), 6.30 (1H, d), 5.87 (2H, d), 4.06 (2H, q), 3.48 (1H, m), 3.05 (1H, m), 3.00 (3H, s), 2.95-2.75 (3H, m), 2.69 (1H, dd), 2.48 (1H, dd), 2.32 (1H, m), 1.16 (3H, s).

### Step 7. (+/−)-[5-(4-Chlorobenzyl)-4-(methylsulfonyl)-5,6,7,8-tetrahydrocyclopenta[4,5]pyrrolor[3,2-c]-pyridin-6-yl]acetic acid

To a 0.088 M solution of the previous ester in THF:MeOH (2:1) at room temperature was added 3.85 equiv of a 1N NaOH aqueous solution. After 2 h, the reaction mixture was poured into a separatory funnel containing NH₄Cl/EtOAc. The layers were separated and the aqueous phase was extracted twice with EtOAc. The combined organic layer was washed with brine, dried over anhydrous Na₂SO₄, filtered and evaporated to dryness. The crude solid was swished in hexanes/CH₂Cl₂ to give the title acid as an off-white solid. ¹H NMR (dmso-d₆) δ 12.32 (1H, br s), 8.99 (1H, s), 8.68 (1H, s), 7.36 (2H, d), 6.82 (2H, d), 6.09 (1H, d), 5.71 (1H, d), 3.23 (1H, m), 3.14 (3H, s), 2.92 (1H, m), 2.79 (1H, m), 2.66 (1H, m), 2.50 (1H, m), 2.25 (1H, m), 2.16 (1H, m). ¹³CNMR (dmso-d₆) δ 172.8, 151.4, 146.6, 141.8, 138.4, 137.2, 131.9, 128.8, 127.4, 122.6, 121.7, 120.4, 49.6, 44.5, 37.9, 35.7, 35.1, 22.4. MS (+APCI) m/z 421.2, 419.0 (M+H)⁺.

### EXAMPLE 2 A

### (−)-[5-[(1S)-1-(4-Chlorophenyl)ethyl]-4-(methylsulfonyl)-5,6,7,8-tetrahydrocyclopenta[4,5]pyrrolo]3,2-c]pyridin-6-yl]acetic acid (Diastereomer A)

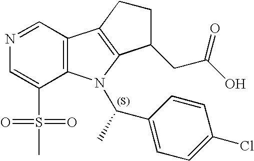

### Step 1. Ethyl [5-[(1S)-1-(4-chlorphenyl)ethyl]-4-(methylsulfonyl)-5,6,7,8-tetrahydrocyclopenta -[4,5]pyrrolo[3,2-c]pyridin-6-yl]acetate (Ethyl ester of Diastereomer A and Ethyl ester of Diastereomer B)

Following the coupling procedure described in example 1, step 6, ethyl [4-(methylsulfonyl)-5,6,7,8-tetrahydrocyclopenta[4,5] pyrrolo[3,2-c]pyridin-6-yl]acetate (example 1, step 5) was reacted with (1R)-1-(4-chlorophenyl)ethanol. The resulting material was purified by flash chromatography on silica gel eluting with a gradient from 100% hexanes to 100% EtOAc. Retention factor on silica gel with 100% EtOAc of the two diastereomers were 0.40 and 0.35. The title compounds were obtained as a less polar diastereomer with >98% de and a more polar diastereomer with >95% de.

Ethyl ester of Diastereomer A (Less polar diastereomer on silica gel eluting with EtOAc): >98% de. Retention factor on Silica gel=0.40 (100% EtOAc). ¹H NMR (acetone-d₆) δ 8.97 (1H, s), 8.87 (1H, s), 7.37 (2H, d), 7.08 (2H, d), 6.98 (1H, q), 4.09 (2H, m), 3.41 (3H, s), 3.00-2.70 (4H, m), 2.49 (1H, dd), 2.43 (1H, m), 2.22 (1H, dd), 2.11 (3H, d), 1.20 (3H, t).

Ethyl ester of Diastereomer B (More polar diastereomer on silica gel eluting with EtOAc): >95% de. Retention factor on Silica gel=0.35 (100% EtOAc). ¹H NMR (acetone-d₆) δ 8.95 (1H, s), 8.87 (1H, s), 7.59 (2H, d), 7.45 (2H, d), 7.02 (1H, q), 4.02 (2H, m) 3.49 (1H, s), 3.47 (3H, s), 2.90-2.75 (3H, m), 2.70 (1H, m), 2.19 (1H, dd), 1.89 (3H, d), 1.76 (1H, dd), 1.19 (3H, t).

### Step 2. (−)-[5-[(1S)-1-(4-Chlorophenyl)ethyl]-4-(methylsulfonyl)-5,6,7,8-tetrahydrocyclopenta -[4,5]pyrrolo[3,2-c]pyridin-6-yl]acetic acid (Diastereomer A)

The title compound was prepared from the Ethyl ester of Diastereomer A (less polar diastereomer) of the previous step according to the procedure described in example 1, step 7, to yield a white solid. [α]D²²=−121.4° (c 0.5, MeOH). ¹H NMR (acetone-d₆) δ 10.80 (1H, br s), 8.95 (1H, s), 8.84 (1H, s), 7.34 (2H, d), 7.03 (2H, d), 6.96 (1H, q), 3.37 (3H, s), 3.00-2.70 (4H, m), 2.45 (1H, dd), 2.42 (1H, m), 2.22 (1H, dd), 2.10 (3H,d). MS(+APCI) m/z 435.3, 433.3 (M+H)⁺.

### EXAMPLE 2 B

### (−)-[5-[(1S)-(4-Chlorophenyl)ethyl]-4-(methylsulfonyl)-5,6,7,8-tetrahydrocyclopenta[4,5]pyrrolo[3,2-c]pyridin-6-yl]acetic acid (Diastereomer B)

The title compound was prepared from the ethyl ester of Diastereomer B (more polar diastereomer) of the example 2 A, step 1, according to the procedure described in example 1, step 7, to yield an off-white solid. [α]D²²=−232.6° (c 0.5, MeOH). ¹H NMR (acetone-d₆) δ 10.52 (1H, s), 8.93 (1H, s), 8.84 (1H, s), 7.51 (2H, d), 7.41 (2H, d), 7.00 (1H, q), 3.48 (1H, m), 3.42 (3H, s), 2.90-2.70 (3H, m), 2.68 (1H, m), 2.17 (1H, m), 1.88 (3H, d), 1.75 (1H, dd). MS (+APCI) m/z 435.3, 433.4 (M+H)⁺.

1. A compound of formula I: 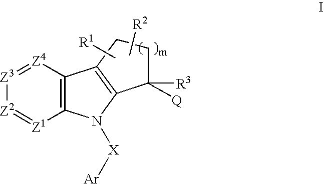
and pharmaceutically acceptable salts and hydrates thereof, wherein:
Ar is aryl or heteroaryl each optionally substituted with one to four groups independently selected from R^{g};
Q is —A—Q′;
A is selected from (1) C₁₋₃alkyl optionally substituted with one to four halogen atoms or with one to two CF₃ groups, (2) O(CH₂)₁₋₂, and (3) S(O)ₙ(CH₂)₁₋₂;
Q′ is selected from COOH, CONR^{a}R^{b}, C(O)NHSO₂R^{c}, SO₂NHR^{a}, SO₃H, PO₃H₂, and tetrazolyl;
one of Z¹, Z², Z³ or Z⁴ is N or N→O, and the others are independently selected from CH and C—R^{g};
X is selected from —(CR^{d}R^{e})ₐ—W—(CR^{d}R^{e})_{b}, phenylene, C₁₋₆cycloalkylidene and C₃₋₆cycloalkylene, wherein a and b are integers 0-1 such that the sum of a and b equals 0, 1 or 2, and W is a bond, —SO₂—, —C(O)—, —CH(OR^{a})—, —C(O)O—, —C(O)NR^{a}—, —CR^{d}═CR^{e}— or —C≡C—;
R¹ is selected from H, CN, OR^{a}, —S(O)ₙC₁₋₆alkyl and C₁₋₆alkyl optionally substituted with one to six groups independently selected from halogen, OR^{a} and —S(O)ₙC₁₋₆alkyl;
R² is H or C₁₋₆alkyl optionally substituted with one to six halogen; or
R¹ and R² together represent an oxo; or
R¹, R² and the atom(s) to which they are attached taken together form a 3- or 4-membered ring containing 0 or 1 heteroatom selected from NR^{f}, S, and O optionally substituted with one or two groups selected from F, CF₃ and CH₃;
R³ is H or C₁₋₆alkyl optionally substituted with one to six groups independently selected from —OR^{a} and halogen;
R^{a} and R^{b} are independently selected from H, C₁₋₁₀alkyl, C₂₋₁₀alkenyl, C₂₋₁₀alkynyl, Cy and Cy-C₁₋₁₀alkyl-, wherein said alkyl, alkenyl, alkynyl and Cy are optionally substituted with one to six substituents independently selected from halogen, amino, carboxy, C₁₋₄alkyl, OH, C₁₋4alkoxy, aryl, heteroaryl, aryl-C₁₋₄alkyl-, hydroxy, CF₃, —OC(O)C₁₋₄alkyl, —OC(O)NRⁱR^{j}, and aryloxy; or
R^{a} and R^{b} together with the atom(s) to which they are attached form a heterocyclic ring of 4 to 7 members containing 0-2 additional heteroatoms independently selected from oxygen, sulfur and N—R^{f};
R^{c} is selected from C₁₋₆alkyl optionally substituted with one to six halogen, aryl and heteroaryl, wherein said aryl and heteroaryl are optionally substituted with halogen, —OC₁₋₆alkyl, C₁₋₆alkyl and wherein said alkyl is optionally substituted with one to six halogen;
R^{d} and R^{e} are independently H, halogen, aryl, heteroaryl, C₁₋₆alkyl or haloC₁₋₆alkyl;
R^{f} is selected from H, C₁₋₆alkyl, haloC₁₋₆alkyl, Cy, -C(O)C₁₋₆alkyl, —C(O)haloC₁₋₆ alkyl, and —C(O)-Cy;
R^{g} is selected from (1) halogen, (2) CN, (3) C₁₋₆alkyl optionally substituted with one to eight groups independently selected from aryl, heteroaryl, halogen, NR^{a}R^{b}, C(O)R^{a}, C(OR^{a})R^{a}R^{b}, SR^{a} and OR^{a}, wherein aryl, heteroaryl and alkyl are each optionally substituted with one to six groups independently selected from halogen, CF₃, and COOH, (4) C₂₋₆alkenyl optionally substituted with one to six groups independently selected from halogen and OR^{a}, (5) Cy, (6) C(O)R^{a}, (7) C(O)OR^{a}, (8) CONR^{a}R^{b}, (9) OCONR^{a}R^{b}, (10) OR^{a}, (11) SH, (12) —S(O)ₙC₁₋₆alkyl, wherein alkyl is optionally substituted with one to six substituents selected from halogen, aryl, heteroaryl, OH, and OC(O)R^{a}, (13) —S(O)ₙaryl, (14) —S(O)ₙheteroaryl, (15) —NR^{a}S(O)ₙR^{b}, (16) —NR^{a}R^{b}, (17) —NR^{a}C(O)R^{b}, (18) —NR^{a}C(O)OR^{b}, (19) —NR^{a}C(O)NR^{a}R^{b}, (20) —S(O)ₙNR^{a}R^{b}, (21) NO₂, (22) C₅₋₈cycloalkenyl; wherein Cy is optionally substituted with one to eight groups independently selected from halogen, C(O)R^{a}, OR^{a}, C₁₋₃alkyl, aryl, heteroaryl and CF₃;
Rⁱ and R^{j} are independently selected from hydrogen, C₁₋₁₀alkyl, Cy and Cy-C₁₋₁₀alkyl-; or
Rⁱ and R^{j} together with the nitrogen atom to which they are attached form a ring of 5 to 7 members containing 0-2 additional heteroatoms independently selected from oxygen, sulfur and N—R^{f};
Cy is selected from heterocyclyl, aryl, and heteroaryl;
m is 1,2or 3; and
n is 0, 1 or 2.

2. A compound of claim 1 wherin Q is CH₂CO₂H.

3. A compound of claim 1 wherein X-Ar is —(CR^{d}R^{e})ₐ—(CR^{d}R^{e})_{b}—aryl, —SO₂-aryl or —C(O)-aryl, wherein said aryl is naphthyl or phenyl optionally substituted with 1 to 2 groups selected from R^{g}.

4. A compound of claim 1 wherein X-Ar is benzyl or α-methylbenzyl wherein the phenyl moiety is substituted with one to three chlorine atoms.

5. A compound of claim 1 wherein Z³ is nitrogen and Z¹, Z² and Z⁴ are independently selected from CH and CR^{g}.

6. A compound of claim 1 wherein Z³ is nitrogen and one of Z¹, Z² and Z⁴ is CR^{g} and the others are CH.

7. A compound of claim 1 wherein Z³ is nitrogen, Z¹ is C—SO₂—C₁₋₃alkyl, Z² and Z⁴ are each CH.

8. A compound of claim 1 wherein m is 1 or 2.

9. A compound of claim 1 wherein R¹, R² and R³ are each hydrogen, or R¹ and R² together is oxo, and R³ is hydrogen.

10. A compound of claim 1 having the formula Ia: 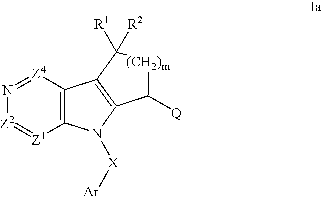
wherein Ar, Q, X, Z¹, Z², Z⁴, R¹, R² and m are as defined in claim 1.

11. A compound of claim 10 wherein Q is CH₂CO₂H.

12. A compound of claim 10 wherein X is CH₂ or CH(CH₃).

13. A compound of claim 10 wherein Ar is phenyl optionally substituted with one to three groups selected from R^{g}.

14. A compound of claim 10 wherein Ar is phenyl optionally substituted with one to three halogen atoms.

15. A compound of claim 10 wherein Z² and Z⁴ are each CH.

16. A compound of claim 1 having the formula Ib: 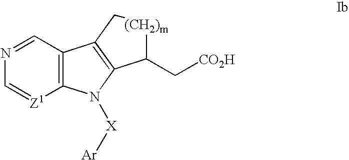
wherein Z¹ and m are as defined in claim 1; Ar is phenyl optionally substituted with one or two R^{g} groups, and X is CH₂ or CH(CH₃).

17. A compound of claim 16 wherein Z₁ is C—SO₂—C₁₋₃alkyl.

18. A compound of claim 16 wherein Ar is phenyl substituted with one or two halogen atoms.

19. A compound of claim 16 wherein Z¹ is C—SO₂—C₁₋₃alkyl and Ar is phenyl substituted with one or two halogen atoms.

20. A pharmaceutical composition comprising a compound of claim 1 and a pharmaceutically acceptable carrier.

21. The composition of claim 20 further comprising a second active ingredient selected from an antihistamine, a leukotriene antagonist and a leukotriene biosynthesis inhibitor.

22. A method for the treatment of prostaglandin D2 mediated diseases or conditions which comprises administering to a patient in need of such treatment a therapeutically effective amount of a compound of claim 1.

23. A method of claim 22 wherein said prostaglandin D2 mediated disease or condition is selected from nasal congestion, allergic rhinitis, asthma and flushing induced by niacin.

24. (canceled)

25. (canceled)

26. (canceled)

27. (canceled)
